(19) United States
(12) Patent Application Publication
(10) Pub. No.: US 2025/0009612 A1
(43) Pub. Date: Jan. 9, 2025

(54) PRODUCT COMPOSITION COMPRISING A DISCRETE PARTICLE AND A NON-AQUEOUS BASE

(71) Applicant: The Procter & Gamble Company, Cincinnati, OH (US)

(72) Inventors: Gemma Zoe BRAGANZA, Singapore (SG); Shikhar GUPTA, Singapore (SG)

(21) Appl. No.: 18/757,690

(22) Filed: Jun. 28, 2024

Related U.S. Application Data
(60) Provisional application No. 63/510,999, filed on Jun. 29, 2023.

(51) Int. Cl. A61K 8/04; A61Q 5/12

(52) U.S. Cl. CPC A61K 8/04; A61Q 5/12; A61K 2800/31; A61K 2800/33

(57) ABSTRACT

A personal care composition that contains discrete particles of a mixture composition and a non-aqueous, fluid base composition. The mixture composition is made from a surfactant, a high melting point fatty compound, and other optional ingredients. The non-aqueous base is at least 75% of a fluid that is not a hydrogen donor.

### FIELD

The present disclosure relates generally to a personal care composition containing discrete particles of a mixture composition and a non-aqueous base. More specifically, the present disclosure relates to discrete particles of a mixture composition comprising a surfactant and a high melting point fatty compound dispersed in a non-aqueous base comprising at least 75% of a fluid that is not a hydrogen donor.

### BACKGROUND

A variety of approaches have been developed to condition the hair. A common method of providing conditioning benefit is through the use of conditioning agents such as cationic surfactants and polymers, high melting point fatty compounds, low melting point oils, silicone compounds, and mixtures thereof. Most of these conditioning agents are known to provide various conditioning benefits by deposition of the traditional gel network within an aqueous chassis.

Another approach widely used by consumers is to apply hair oils or other anhydrous products to wet or dry hair to improve their manageability and softness. But these hair oils do not include the benefits provided by the cationic surfactants and fatty compounds.

Thus, there is a continuing need for non-aqueous hair care compositions that can still deliver the conditioning benefits that consumers want and expect.

### SUMMARY

A product composition comprising a discrete particle of a mixture composition, wherein the mixture composition comprises a surfactant and a high melting point fatty compound; and a non-aqueous base, wherein the non-aqueous base comprises at least 75% of a fluid that is not a hydrogen donor.

### BRIEF DESCRIPTION OF THE FIGURES

FIG. 1 is a photograph of the initial appearance of an inventive composition.

FIG. 2 is a photograph of the appearance of an inventive composition after the 4 Week Stability Test.

FIG. 3 is a photograph of the appearance of a comparative composition after the 4 Week Stability Test.

### DETAILED DESCRIPTION

Reference within the specification to “embodiment(s)” or the like means that a particular material, feature, structure and/or characteristic described in connection with the embodiment is included in at least one embodiment, optionally a number of embodiments, but it does not mean that all embodiments incorporate the material, feature, structure, and/or characteristic described. Furthermore, materials, features, structures and/or characteristics may be combined in any suitable manner across different embodiments, and materials, features, structures and/or characteristics may be omitted or substituted from what is described. Thus, embodiments and aspects described herein may comprise or be combinable with elements or components of other embodiments and/or aspects despite not being expressly exemplified in combination, unless otherwise stated or an incompatibility is stated.

All ingredient percentages described herein are by weight of the cosmetic composition, unless specifically stated otherwise, and may be designated as “wt %.” All ratios are weight ratios, unless specifically stated otherwise. All such percentages or weights as they pertain to listed ingredients are based on the active level and, therefore, do not include carriers or by-products that may be included in commercially available materials. The number of significant digits conveys neither a limitation on the indicated amounts nor on the accuracy of the measurements. Unless otherwise indicated, all measurements are understood to be made at approximately 25° C. and at ambient conditions, where “ambient conditions” means conditions under 1 atmosphere of pressure and at 50% relative humidity. All ranges are inclusive and combinable. For example, all numeric ranges are inclusive of narrower ranges, and delineated upper and lower range limits are interchangeable to create further ranges not explicitly delineated.

The compositions of the present invention can comprise, consist essentially of, or consist of, the essential components as well as optional ingredients described herein. As used herein, “consisting essentially of” means that the composition or component may include additional ingredients, but only if the additional ingredients do not materially alter the basic and novel characteristics of the claimed compositions or methods. As used in the description and the appended claims, the singular forms “a,” “an,” and “the” are intended to include the plural forms as well, unless the context clearly indicates otherwise.

“About” modifies a particular value by referring to a range of plus or minus 20% or less of the stated value (e.g., plus or minus 15% or less, 10% or less, or even 5% or less).

“Mixture” means a simple combination of materials and any compounds that may result from their combination.

“Molecular weight” means the weight average molecular weight, unless otherwise stated. Molecular weight can be measured using industry standard method, gel permeation chromatography (“GPC”).

“Non-aqueous” means a material or composition contain less than 10% water (e.g., less than 5%, 3%, 1%, or 0.5%, or in some cases 0% water).

“Hydrogen donor” or “hydrogen bond donor” means a compound or functional group that has a hydrogen atom bonded to an oxygen, nitrogen, fluorine, chlorine, or sulfur atom.

“Solid” means that the anhydrous discrete particle does not conform to the shape of the container in which the particle is held at 25° C.

“Substantially free of” means 2% or less (e.g., less than 1%, 0.5% or even less than 0.1%) of a stated material or ingredient. “Free of” means no detectable amount of the stated material or ingredient.

### Personal Care Composition

Conventional aqueous-based hair care products such as shampoos and conditioners generally deliver hair care cleaning and conditioning benefits that consumers expect. In some instances, consumers are known to apply conditioning oils to hair in a pre-wash and/or post-wash step. Pre-wash oil application is believed to nourish hair and protect it during the wash process. Post-wash oil application may be done for manageability and styling.

The personal care compositions disclosed herein contain discrete particles of a mixture composition dispersed in a non-aqueous carrier. The present products can provide more flexibility than conventional gel network compositions, improved visual/aesthetic appearance, improved stability, especially when using smaller amounts of actives such as surfactants and high melting point fatty compounds, and improved deposition of benefit agents. Desirably, the stability improvement can be obtained without the use of a thickening polymer.

The personal care product herein can be a hair care product, a body care product composition, a facial skin care product, and mixtures thereof. In some instances, the personal care product is a hair conditioning product (e.g., a conditioning shampoo or a conditioner). The personal care compositions herein can be in the form of rinse-off products or leave-on products and can be formulated in a wide variety of product forms, including but not limited to oils, gels, emulsions, mousses and sprays. The personal care compositions herein may be used for the non-therapeutic treatment of hair such as, for example, cleaning and/or conditioning. The personal care composition may be particularly suitable for use as a hair conditioner, especially a leave-on hair oil.

In some instances, the personal care product can be free of or substantially free of detersive surfactants. The detersive surfactants herein are those selected from anionic surfactants, zwitterionic surfactant, amphoteric surfactant, and combinations thereof.

### Mixture Composition

The mixture composition herein comprises a surfactant, a high melting point fatty compound and, optionally, a benefit agent. The surfactant and high melting point fatty compound are described in more detail below. The mixture composition may be added to a product composition at a level of 0.01% to 70% (e.g., 0.01% to 50% or 0.05% to 30%), in view of having discrete particles of the mixture composition in the non-aqueous base composition and in the product composition, and also in view of providing at least one the followings: more flexibility and stability in the product composition; improved deposition of benefit agents; and improved visual/aesthetic appearance.

The surfactant(s) and the high melting point fatty compound(s) may be present in the mixture composition, with or without other ingredients, at 10% to 99.9% (e.g., 20% to 99.9%, 40% to 99.9%, 60% to 99.9%, 80% to 99.9% or to even 100%), based on the weight of the mixture composition, in view of having discrete particles of the mixture composition in the non-aqueous base composition and in the personal care composition while containing other ingredients such as benefit agents, and also in view of providing at least one the followings: more flexibility in the product composition; improved stability in the product composition; improved deposition of benefit agents; and improved visual/aesthetic appearance.

Other than the surfactants and high melting point fatty compounds, when the mixture composition contains any liquid such as water-insoluble, water-miscible, and water-soluble liquids and water, it is also preferred to control the level of such liquids, so that the total liquid level in the mixture composition can be up to 30%, 40%, 50%, 60% or even up to 92%, by weight of the mixture composition, in view of having discrete particle of the mixture composition in the non-aqueous base composition and in the product composition.

When the liquid is water insoluble liquid such as a silicone oil, a water miscible liquid such as propylene glycol or glycerin, or a water-soluble liquids such as isopropyl alcohol (IPA) or ethanol, such liquid can be contained in the mixture composition at a level by weight of the mixture composition of up to 30%, 40%, 50%, 60%, or even up to 90%.

When the mixture composition contains water, it may be desirable to limit the level of water to less than 50% (e.g., less than 25%, 15%, 10%, or even less than 8%), by weight of the mixture composition, in view of having discrete particles of the mixture composition in the non-aqueous base composition and in the product composition.

The surfactant and the high melting point fatty compound may be included in the mixture composition such that the weight ratio of the surfactant to the high melting point fatty compound is in the range of 1:1 to 1:30 (e.g., 1:10 to 1:30 or 1:10 to 1:20) to provide a suitable conditioning benefit.

### Benefit Agent for the Mixture Composition

The mixture composition may further comprise a benefit agent in addition to the surfactant and the high melting point fatty compound. The benefit agent may be present in the mixture composition at a level by the weight of the mixture composition, of 0.1% to 90% (e.g., 0.3% to 60%, 0.5% to 40%, or even 0.5% to 30%) in view of providing benefits from the benefit agents and in view of having discrete particle of the mixture composition in the non-aqueous composition and in the product composition.

Some non-limiting examples of benefit agents that may be suitable for use herein include silicone compounds, perfumes (e.g., conventional perfumes or perfume micro capsules (PMC), in which perfume is encapsulated by a polymeric outer layer), coloring agents (e.g., pigments or dyes that provide the discrete particle with a color that is different from the color of the non-aqueous base composition), agents which are incompatible to at least one ingredient contained in the mixture composition, discrete particle and/or non-aqueous base composition (“incompatible agents”), and mixtures thereof. In some embodiments the incompatible agent may be a solid mineral or chemical substance that has a high ionic strength and/or high surface charge and tends to cause agglomeration and/or crystallization (e.g., mica, salicylic acid, zinc pyrithione, and/or an organic oil material that undesirably interacts with a gel network component such as, for example, hexyldecanol and isostearyl isostearate).

When the mixture composition contains a silicone or perfume benefit agent, the mixture compositions may be contained in the personal care composition at a level of 0.1% to 70% (e.g., 0.5% to 50%, 1% to 30%, or even 2% to 20%). When the mixture composition contains a coloring agent or mica benefit agent, the mixture compositions may be contained in the product composition at 0.01% to 50% (e.g., 0.01% to 30%, 0.01% to 20% or 0.05% to 10%).

### Non-Aqueous Base

The personal care compositions herein include a non-aqueous base, wherein at least 75% (e.g., at least 80%, 85%, 90% or even 95% or more) of the non-aqueous base is a fluid(s) that is not a hydrogen donor. In some embodiments, the fluid of the non-aqueous base may be 100% of a fluid that is not a hydrogen donor, for example, a single fluid material that is not a hydrogen donor.

Previously, combinations of a surfactant and a high melting point fatty compound as a discrete particle within a base composition were done in an aqueous carrier. One reason was it was believed that the particles would not be able to be maintained as discrete particles, but that they would disintegrate too quickly due to the relatively high solubility of the fatty compound in the non-aqueous carrier.

With the choice of certain non-aqueous bases, it has surprisingly been found that the discrete particle can maintain its integrity. The present inventors have now discovered that when the non-aqueous base is substantially made up of a material that is not a hydrogen donor, the discrete particle described herein is not dissolved within the non-aqueous base. The choice of a non-aqueous base that comprises at least 75% of a fluid that is not a hydrogen donor, when combined with the discrete particle, allows the particle to resist disintegration over time. For example, the particle will not exhibit any significant disintegration within 4 weeks if placed in elevated temperature conditions (e.g., 40° C.).

The non-aqueous base composition comprises at least 75% of a fluid that is not a hydrogen donor, and the fluid may be an oil. The present inventors have found that not all oils are able to be part of a conditioning formulation that does not disintegrate the discrete particles. They have discovered that oils that are not a hydrogen donor are able to be formulated successfully. Also relevant is the AlogP of the fluid. Fluids that successfully formulate while not disintegrating the discrete particle (as defined by the 4 Week Stability Test Method) include those that have an AlogP of at least 6.

The fluid of the non-aqueous base may be an oil, in some cases a low melting point oil, defined herein as oils having a melting point of 27° C. or lower (e.g., 25° C. or lower). Some non-limiting examples of a low melting point oils useful herein include 1-decanol, isohexadecane, isododecane, triethylhexanoin, isodecyl oleate, decyl oleate, silicone quaternium-26, dimethicone, hydrocarbons having 10 to 40 carbon atoms, unsaturated fatty alcohols having 10 to 30 carbon atoms such as oleyl alcohol; unsaturated fatty acids having 10 to 30 carbon atoms; fatty acid derivatives; fatty alcohol derivatives; ester oils (e.g., pentaerythritol ester oils such as pentaerythritol tetraisostearate), trimethylol ester oils, citrate ester oils, glyceryl ester oils, poly α-olefin oils such as polydecenes; and mixtures thereof.

Additional appropriate oils may include triglycerides, such as caprylic capric triglyceride or vegetable oils such as coconut oil, soybean oil, rapeseed oil, cocoa butter, olive oil, palm oil, rice bran oil, and mixtures thereof.

Additional fluids of the non-aqueous base may include, but are not limited to, hydrocarbons, branched hydrocarbons, more specifically an alkane, such as C13-C16 isoparaffin, isohexadecane, pentadecane, isododecane, C15-19 alkane, C9-12 alkane, C13-15 alkane, C14-22 alkane, coconut alkanes.

Also appropriate for use as the non-aqueous fluid may be an ester, including, but not limited to, isobutyl palmitate, isodecyl oleate, decyl oleate, oleyl oleate, triethylhexanoin, isopropyl isostearate, isostearyl isostearate, myristyl myristate, isopropyl myristate, propylene glycol dicaprate, diethylhexyl adipate.

The fluid of the non-aqueous base may, or may not, be or comprise a silicone compound. It is believed that the silicone compound can provide smoothness and softness on dry hair. The silicone compounds herein can be used at levels by weight of the composition, or by weight of the non-aqueous base, of 0.1%-99%.

### Benefit Agent for the Non-Aqueous Base Composition

The non-aqueous base composition may include 0.05% to 60% (e.g., 0.1% to 30%, 0.1% to 20%, or 0.1% to 10%), by the weight of the non-aqueous base composition, of a benefit agent. The benefit agent may be present in the personal care composition at 0.05% to 30% (e.g., 0.1% to about 15%, 0.1% to 10%, or 0.1% to about 7%), by weight of the personal care composition.

Some non-limiting examples of benefit agents that may be suitable for use herein include silicone compounds, perfumes (e.g., conventional perfumes or perfume micro capsules (PMC), in which perfume is encapsulated by a polymeric outer layer), coloring agents (e.g., pigments or dyes that provide the non-aqueous base with a color that is different from the color of the discrete particles), incompatible agents, and mixtures thereof.

Depending on the type of the benefit agent, when containing the benefit agent, the personal care composition may provide at least one of the following benefits: improved deposition of benefit agents and improved stability when containing incompatible agents.

### Discrete Particle

The personal care compositions herein contain discrete particles of the mixture composition. The discrete particles are dispersed in the non-aqueous base composition and can be visually observed as discrete particle in final product composition visually. For example, the particles can be observed by an unaided human eye with 20/20 vision at a distance of 25 cm in typical day time lighting.

The discrete particles are contained in the product composition at a level by weight of the product composition, of 0.1% to 70% (e.g., 0.1% to 50% or 0.1% to 30%), in view of providing at least one the followings: more flexibility in the product composition; improved stability in the product composition; improved deposition of benefit agents; and improved visual/aesthetic appearance.

The discrete particles herein can be in any shape, for example, spherical shape, rectangular shape, diamond shape, planar (flake-shaped) or fiber-shaped (e.g., an elongated particle with an aspect ratio of great than 3:1).

Before adding to the non-aqueous base, the discrete particle is 100% of the mixture composition, i.e., consists of the mixture composition and is solid. However, the particle is not an emulsion and is not coated or encapsulated by, for example, polymers. The surfactants and high melting point fatty compound(s) are present in the discrete particle, with or without other ingredients, at a level, by weight of the discrete particle, of 10% to 100% (e.g., 20% to about 99%, 40% to 95%, 60% to 90%, or even 80% to 85%), in view of having discrete particles of the mixture composition in the non-aqueous base composition and in the product composition.

The discrete particles herein are different from swellable silicone elastomer and swellable thickening polymers and can be free or substantially free of such swellable silicone elastomer and swellable thickening polymer. In the present invention, “the discrete particle and the mixture composition being substantially free of swellable silicone elastomer and swellable thickening polymer” means that: the discrete particle and the mixture composition are free of swellable silicone elastomer and swellable thickening polymer; or, if the discrete particle and the mixture composition contains swellable silicone elastomer and swellable thickening polymer, the level of such swellable silicone elastomer and swellable thickening polymer is very low.

### Second Discrete Particle

The product composition of the present invention may comprise a second discrete particle, in addition to the above discrete particle of the present invention which comprises the mixture comprising the surfactant, high melting point fatty alcohol, and benefit agent. The second discrete particle useful herein comprises the mixture excluding the benefit agent, i.e., a mixture comprising a surfactant and a high melting point fatty compound. The components and the properties of the second discrete particles are the same as those described for the above discrete particle of the present invention, except for the inclusion of benefit agents.

### Cationic Surfactant

The mixture composition and/or base composition may include a cationic surfactant. Some non-limiting examples of a cationic surfactant include mono-long alkyl quaternized ammonium salt, a combination of mono-long alkyl quaternized ammonium salt and di-long alkyl quaternized ammonium salt, mono-long alkyl amine, and a combination of mono-long alkyl amine and di-long alkyl quaternized ammonium salt.

### Long Alkyl Quaternized Ammonium Salt

The cationic surfactant may be a mono- or di-long alkyl quaternized ammonium salts having one or two, respectively, long alkyl chain(s) which has from 12 to 30 carbon atoms (e.g., 16 to 24 carbon atoms or 18 to 22 carbon atoms). The remaining groups attached to nitrogen are independently selected from an alkyl group of from 1 to about 4 carbon atoms or an alkoxy, polyoxyalkylene, alkylamido, hydroxyalkyl, aryl or alkylaryl group having up to about 4 carbon atoms. An exemplary chemical structure for a long alkyl quaternized ammonium salt is described in U.S. Pat. No. 11,633,338. When used, a di-long alkyl quaternized ammonium salt may be combined with a mono-long alkyl quaternized ammonium salts or mono-long alkyl amine salt, at the weight ratio of 1:1 to 1:5 (e.g., 1:1.2 to 1:5 or 1:1.5 to 1:4), in view of stability in rheology and conditioning benefits.

Some nonlimiting examples of mono-long alkyl quaternized ammonium salt cationic surfactants that may be suitable for use herein include behenyl trimethyl ammonium salt; stearyl trimethyl ammonium salt; cetyl trimethyl ammonium salt; and hydrogenated tallow alkyl trimethyl ammonium salt. Some nonlimiting examples of di-long alkyl quaternized ammonium salt cationic surfactants that may be suitable for use herein include dialkyl (14-18) dimethyl ammonium chloride, ditallow alkyl dimethyl ammonium chloride, dihydrogenated tallow alkyl dimethyl ammonium chloride, distearyl dimethyl ammonium chloride, and dicetyl dimethyl ammonium chloride.

### Long Alkyl Amine

The cationic surfactants herein may include a long alkyl amine cationic surfactant having one long alkyl chain of 12 to 30 carbon atoms (e.g., 16 to 24 carbon atoms or 18 to 22 carbon atoms). The long alkyl amines herein also include mono-long alkyl amidoamines. Primary, secondary, and tertiary fatty amines may be useful. Particularly useful are tertiary amido amines having an alkyl group of 12 to 22 carbons. Exemplary tertiary amido amines include: stearamidopropyldimethylamine, stearamidopropyldiethylamine, stearamidoethyldiethylamine, stearamidocthyldimethylamine, palmitamidopropyldimethylamine, palmitamidopropyldiethylamine, palmitamidoethyldicthylamine, palmitamidocthyldimethylamine, behenamidopropyldimethylamine, behenamidopropyldiethylamine, behenamidoethyldiethylamine, behenamidoethyldimethylamine, arachidamidopropyldiethylamine, arachidamidopropyldimethylamine, arachidamidoethyldiethylamine, arachidamidocthyldimethylamine, diethylaminoethylstearamide. Some additional non-limiting examples of amines that may be useful herein are disclosed in U.S. Pat. No. 4,275,055.

These amines may be used in combination with acids such as l-glutamic acid, lactic acid, hydrochloric acid, malic acid, succinic acid, acetic acid, fumaric acid, tartaric acid, citric acid, l-glutamic hydrochloride, maleic acid, and mixtures thereof; more preferably l-glutamic acid, lactic acid, citric acid, at a molar ratio of amine to acid of 1:0.3 to 1:2 (e.g., 1:0.4 to about 1:1).

### High Melting Point Fatty Compound

The mixture composition and, optionally, the base composition contain a high melting point fatty compound for providing hair conditioning benefits such as hair friction reduction, case of product spreading, and/or case of hair detangling, compared to compositions containing no high melting point fatty compounds and/or compared to compositions containing low melting point fatty compounds instead of high melting point fatty compounds. The high melting point fatty compound can also provide improved product stability (e.g., reduced risk of phase separation), compared to compositions containing no high melting point fatty compounds and/or compared to compositions containing low melting point fatty compounds instead of high melting point fatty compounds.

The high melting point fatty compound useful herein have a melting point of 25° C. or higher (e.g., 40° C. or higher, 45° C. or higher, or even 50° C. or higher), but typically less than 90° C. (e.g., less than 80° C., 70° C., or 65° C.), in view of easier manufacturing and easier emulsification. The high melting point fatty compound may be a single compound or as a blend or mixture of two or more fatty compounds. When used as such blend or mixture, the melting point of the blend or mixture of fatty compounds is 25° C. or higher (e.g., 40° C. or higher, 45° C. or higher, or even 50° C. or higher).

Some non-limiting examples of high melting point fatty compounds that may be useful herein include fatty alcohols, fatty acids, fatty alcohol derivatives, fatty acid derivatives, and mixtures thereof. It is to be understood that the exemplified fatty compounds may fall into more than one classification, e.g., some fatty alcohol derivatives can also be classified as fatty acid derivatives. However, a given classification is not intended to be a limitation on that particular compound, but is done so for convenience of classification and nomenclature. Further, it is to be appreciated that, depending on the number and position of double bonds, and length and position of the branches, certain compounds having certain required carbon atoms may have a melting point of less than the above preferred in the present invention. Such compounds of low melting point are not intended to be included in this section. Nonlimiting examples of the high melting point compounds are found in International Cosmetic Ingredient Dictionary, Fifth Edition, 1993, and CTFA Cosmetic Ingredient Handbook, Second Edition, 1992.

Among a variety of high melting point fatty compounds, fatty alcohols may be particularly suitable for use in the personal care products herein, especially those having 14 to 30 carbon atoms (e.g., 16 to about 22 carbon atoms). These fatty alcohols can be saturated or unsaturated and can be straight or branched chain alcohols. Some particularly suitable fatty alcohols include, for example, cetyl alcohol (having a melting point of about 56° C.), stearyl alcohol (having a melting point of about 58-59° C.), behenyl alcohol (having a melting point of about 71° C.), and mixtures thereof. In some embodiments, the fatty alcohol is a mixture of cetyl alcohol and stearyl alcohol, wherein the weight ratio of cetyl alcohol to stearyl alcohol is 1:9 to 9:1 (e.g., 1:4 to 4:1 or 1:2.3 to 1.5:1).

### Silicone Compound

The non-aqueous base composition may include (or consist of) a silicone compound. The silicone compound may have an weight average particle size of 10 nm to 100 μm (e.g., 0.1 μm to 100 μm, 1 μm to 50 μm) in the non-aqueous base composition. The silicone compounds may be present as a single compound or a blend or mixture of two or more silicone compounds. The silicone compound may have a viscosity of 1,000 mPa-s to 2,000,000 mPa·s at 25° C., as measured using a glass capillary viscometer according to Dow Corning Corporate Test Method CTM0004, Jul. 20, 1970. Some non-limiting example of silicone fluids that may be suitable for use herein include polyalkyl siloxanes, polyaryl siloxanes, polyalkylaryl siloxanes, polyether siloxane copolymers, amino substituted silicones, quaternized silicones, and mixtures thereof. In some instances, amino substituted silicones and/or quaternized silicones may be preferred.

Some non-limiting examples of polyalkyl siloxanes include polydimethylsiloxane, polydiethylsiloxane, and polymethylphenylsiloxane. Polydimethylsiloxane, which is also known as dimethicone, is especially preferred. These silicone compounds are available, for example, from the General Electric Company in their Viscasilâ and TSF 451 series, and from Dow Corning in their Dow Corning SH200 series. The above polyalkylsiloxanes are available, for example, as a mixture with silicone compounds having a lower viscosity. Such mixtures have a viscosity of 1,000 mPa-s to 100,000 mPa-s (e.g., 5,000 mPa-s to about 50,000 mPa-s). Such mixtures may include (i) a first silicone compound having a viscosity of 100,000 mPa-s to 30,000,000 mPa-s at 25° C., (e.g., 100,000 mPa-s to 20,000,000 mPa-s); and (ii) a second silicone compound having a viscosity of 5 mPa-s to 10,000 mPa-s at 25° C. (e.g., 5 mPa-s to 5,000 mPa-s. Such mixtures useful herein include, for example, a blend of dimethicone having a viscosity of 18,000,000 mPa-s and dimethicone having a viscosity of 200 mPa-s available from GE Toshiba, and a blend of dimethicone having a viscosity of 18,000,000 mPa-s and cyclopentasiloxane available from GE Toshiba. The silicone compounds useful herein also include a silicone gum, which is a polyorganosiloxane material having a kinematic viscosity at 25° C. of greater than or equal to 1,000,000 centistokes. It is recognized that the silicone gums described herein can also have some overlap with the above-disclosed silicone compounds. This overlap is not intended as a limitation on any of these materials. The silicone gums will typically have a mass molecular weight in excess of about 200,000, generally between 200,000 and 1,000,000. Specific examples include polydimethylsiloxane, poly(dimethylsiloxane methylvinylsiloxane) copolymer, poly(dimethylsiloxane diphenylsiloxane methylvinylsiloxane) copolymer and mixtures thereof. The silicone gums are available, for example, as a mixture with silicone compounds having a lower viscosity. Such mixtures useful herein include, for example, Gum/Cyclomethicone blend available from Shin-Etsu.

In some instances, the silicone compounds may include an amino substituted silicone (aminosilicone) that conforms to the general formula (I):

(R₁)ₐG₃₋ₐ-Si—(—OSiG₂)ₙ-(—OSiG_{b}(R₁)_{2-b})ₘ—O—SiG₃₋ₐ(R₁)ₐ (I)
wherein G is hydrogen, phenyl, hydroxy, or C₁-C₈ alkyl, preferably methyl; a is 0 or an integer having a value from 1 to 3, preferably 1; b is 0, 1 or 2, preferably 1; n is a number from 0 to 1,999; m is an integer from 0 to 1,999; the sum of n and m is a number from 1 to 2,000; a and m are not both 0; R₁ is a monovalent radical conforming to the general formula CqH_{2q}L, wherein q is an integer having a value from 2 to 8 and L is selected from the following groups: —N(R₂)CH₂—CH₂—N(R₂)₂; —N(R₂)₂; —N(R₂)₃A⁻; —N(R₂)CH₂—CH₂—NR₂H₂A⁻; wherein R₂ is hydrogen, phenyl, benzyl, or a saturated hydrocarbon radical, preferably an alkyl radical from about C₁ to about C₂₀; A⁻ is a halide ion.

Particularly suitable amino silicones may include those corresponding to formula (I) wherein m=0, a=1, q=3, G=methyl, n is 1500 to 1700 (e.g., about 1600); and L is —N(CH₃)₂ or —NH₂. Another particularly suitable aminosilicone corresponds to formula (I) wherein m=0, a=1, q=3, G=methyl, n is 400 to 600 (e.g., about 500); and L is —N(CH₃)₂ or —NH₂. Such highly preferred amino silicones can be called as terminal aminosilicones, as one or both ends of the silicone chain are terminated by nitrogen containing group.

The above aminosilicones, when incorporated into the personal care composition or non-aqueous base, can be mixed with solvent having a lower viscosity. Such solvents include, for example, polar or non-polar, volatile or non-volatile oils. Such oils include, for example, silicone oils, hydrocarbons, and esters, for example, selected from the group consisting of non-polar volatile hydrocarbons, volatile cyclic silicones, non-volatile linear silicones, and mixtures thereof. The non-volatile linear silicones useful herein are those having a viscosity of 1 to 20,000 centistokes, preferably from about 20 to about 10,000 centistokes at 25° C. Among the preferred solvents, highly preferred are non-polar, volatile hydrocarbons, especially non-polar, volatile isoparaffins, in view of reducing the viscosity of the aminosilicones and providing improved hair conditioning benefits such as reduced friction on dry hair. Such mixtures have a viscosity of preferably from about 1,000 mPa·s to about 100,000 mPa·s, more preferably from about 5,000 mPa·s to about 50,000 mPa·s.

Other suitable alkylamino substituted silicone compounds include those having alkylamino substitutions as pendant groups of a silicone backbone. Highly preferred are those known as “amodimethicone”. Commercially available amodimethicones useful herein include, for example, BY16-872 available from Dow Corning.

### Silicone Polymer Containing Quaternary Groups

Silicone compounds useful herein include, for example, a Silicone Polymer Containing Quaternary Groups comprising terminal ester groups, having a viscosity up to 100,000 mPa·s and a D block length of greater than 200 D units. Without being bound by theory, this low viscosity silicone polymer provides improved conditioning benefits such as smooth feel, reduced friction, and prevention of hair damage, while eliminating the need for a silicone blend.

Structurally, the silicone polymer is a polyorganosiloxane compound comprising one or more quaternary ammonium groups, at least one silicone block comprising greater than 200 siloxane units, at least one polyalkylene oxide structural unit, and at least one terminal ester group. In one or more embodiments, the silicone block may comprise between 300 to 500 siloxane units.

The silicone polymer is present at 0.05% to 15% (e.g., 0.1% to 10%, 0.15% to 5%, or 0.2% to 4%) by weight of the personal care composition.

The polyorganosiloxane compounds can have the general formulas (Ia) and (Ib):

M-Y—[—(N⁺R₂-T-N⁺R₂)—Y-]ₘ-[—(NR²-A-E-A′—NR²)—Y-]ₖ-M (Ia)

M-Y—[—(N⁺R₂-T-N⁺R₂)—Y-]ₘ-[—(N⁺R²₂-A-E-A′-N⁺R²₂)—Y-]ₖ-M (Ib)
wherein:
m is >0, 0.01 to 100 (e.g., 0.1 to 100, 1 to 100, 1 to 50, 1 to 20, or 1 to 10),
k is 0 or an average value of >0 to 50 (e.g., 1 to 20 or 1 to 10),
M represents a terminal group, comprising terminal ester groups selected from
—OC(O)—Z
—OS(O)₂—Z
—OS(O₂)O—Z
—OP(O)(O—Z)OH
—OP(O)(O—Z)₂
wherein Z is selected from monovalent organic residues having up to 40 carbon atoms, optionally comprising one or more hetero atoms.

A and A′ each are independently from each other selected from a single bond or a divalent organic group having up to 10 carbon atoms and one or more hetero atoms, and
E is a polyalkylene oxide group of the general formula:

[CH₂CH₂O]_{q}—[CH₂CH(CH₃)O]ᵣ—[CH₂CH(C₂H₅)O]ₛ—
wherein q=0 to 200, r=0 to 200, s=0 to 200, and q+r+s=1 to 600.
R² is selected from hydrogen or R,
R is selected from monovalent organic groups having up to 22 carbon atoms and optionally one or more heteroatoms, and wherein the free valencies at the nitrogen atoms are bound to carbon atoms,
Y is a group of the formula:

—K—S—K—and-A-E-A′-or-A′-E-A-,
with S=

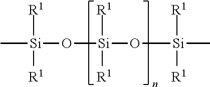

wherein R1═C₁-C₂₂-alkyl, C₁-C₂₂-fluoralkyl or aryl; n=200 to 1000, and these can be identical or different if several S Groups are present in the polyorganosiloxane compound.
K is a bivalent or trivalent straight chain, cyclic and/or branched C₂-C₄₀ hydrocarbon residue which is optionally interrupted by-O—,—NH—, trivalent N, —NR₁—, —C(O)—, —C(S)—, and optionally substituted with-OH, wherein R¹ is defined as above,
T is selected from a divalent organic group having up to 20 carbon atoms and one or more hetero atoms.

The residues K may be identical or different from each other. In the —K—S—K-moiety, the residue K is bound to the silicon atom of the residue S via a C—Si-bond.

Due to the possible presence of amine groups (—(NR²-A-E-A′—NR²)—) in the polyorganosiloxane compounds, they may have protonated ammonium groups, resulting from the protonation of such amine groups with organic or inorganic acids. Such compounds are sometimes referred to as acid addition salts of the polyorganosiloxane compounds.

The molar ratio of the quaternary ammonium groups b) and the terminal ester groups c) may be less than 100:20 (e.g., less than 100:30 or less than 100:50). The ratio can be determined by ¹³C-NMR.

The polyorganosiloxane composition may comprise:
A. at least one polyorganosiloxane compound, comprising a) at least one polyorganosiloxane group, b) at least one quaternary ammonium group, c) at least one terminal ester group, and d) at least one polyalkylene oxide group (as defined before),
B. at least one polyorganosiloxane compound, comprising at least one terminal ester group, different from compound A.

In the definition of component A) it can be referred to the description of the polyorganosiloxane compounds herein. The polyorganosiloxane compound B) can differ from the polyorganosiloxane compound A) in that it does not comprise quaternary ammonium groups. Polyorganosiloxane compounds B) result from the reaction of monofunctional organic acids, in particular carboxylic acids, and polyorganosiloxane containing bisepoxides.

When present in the personal care composition, the weight ratio of compound A) to compound B) may be less than 90:10 (i.e., component B) is present at at least 10 weight percent). The polyorganosiloxane compositions in compound A) may have a molar ratio of the quaternary ammonium groups b) and the terminal ester groups c) of less than 100:10 (e.g., less than 100:15 and less than 100:20).

The silicone polymer has a viscosity at 20° C. and a shear rate of 0.1s⁻¹ (plate-plate system, plate diameter 40 mm, gap width 0.5 mm) of less than 100,000 mPas (100 Pas). In further embodiments, the viscosities of the neat silicone polymers may range from 500 to 100,000 mPas, (e.g., 500 to 70,000 mPas, 500 to 50,000 mPas, or 500 to 20,000 mPas). The viscosities of the neat polymers may range from 500 to 10,000 mPas (e.g., 500 to 5000 mPas) determined at 20° C. and a shear rate of 0.1 s^{-1.}

In the polyalkylene oxide group E of the general formula:

—[CH₂CH₂O]_{q}—[CH₂CH(CH₃)O]ᵣ,[CH₂CH(C₂H₅)O]ₛ—
the q, r, and s indices may be defined as follows:
q=0 to 200 (e.g., 0 to 100, 0 to 50, or even 0 to 20),
r=0 to 200 (e.g., 0 to 100, 0 to 50, or even 0 to 20),
s=0 to 200 (e.g., 0 to 100, 0 to 50, or even 0 to 20), and
q+r+s=1 to 600 (e.g., 1 to 100, 1 to 50, or even 1 to 40).

For polyorganosiloxane structural units with the general formula S:

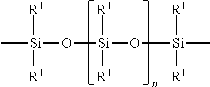

R¹═C₁-C₂₂-alkyl, C₁-C₂₂-fluoralkyl and aryl; n=from 200 to 1000 (e.g., 300 to 500), K (in the group —K—S—K—) is a bivalent or trivalent straight chain, cyclical or branched C₂-C₂₀ hydrocarbon residue which is optionally interrupted by —O—, —NH—, trivalent N, —NR¹—, —C(O)—, —C(S)—, and optionally substituted with —OH.

In some instance, R¹ is C₁-C₁₈ alkyl, C₁-C₁₈ fluoroalkyl and aryl (e.g., C₁-C₁₈ alkyl, C₁-C₆ fluoroalkyl and aryl). In other instances, R¹ is C₁-C₆ alkyl and C₁-C₆ fluoroalkyl. In still other instances R¹ is C₁-C₄ fluoroalkyl and phenyl. It may be particularly desirable for R¹ to be methyl, ethyl, trifluoropropyl and phenyl.

As used herein, the term “C₁-C₂₂ alkyl” means that the aliphatic hydrocarbon groups possess from 1 to 22 carbon atoms which can be straight chain or branched. Methyl, ethyl, propyl, n-butyl, pentyl, hexyl, heptyl, nonyl, decyl, undecyl, isopropyl, neopentyl and 1,2,3-trimethyl hexyl moieties serve as examples.

Further as used herein, the term “C₁-C₂₂ fluoroalkyl” means aliphatic hydrocarbon compounds with 1 to 22 carbon atoms which can be straight chain or branched and are substituted with at least one fluorine atom. Monofluormethyl, monofluoroethyl, 1,1,1-trifluorethyl, perfluoroethyl, 1,1,1-trifluoropropyl, 1,2,2-trifluorobutyl are suitable examples.

Moreover, the term “aryl” means unsubstituted or phenyl substituted once or several times with OH, F, Cl, CF₃, C₁-C₆ alkyl, C₁-C₆ alkoxy, C₃-C₇ cycloalkyl, C₂-C₆ alkenyl or phenyl. Aryl may also mean naphthyl.

For the embodiments of the polyorganosiloxanes, the positive charges resulting from the ammonium group(s), are neutralized with inorganic anions such as chloride, bromide, hydrogen sulfate, sulfate, or organic anions, like carboxylates deriving from C₁-C₃₀ carboxylic acids, for example acetate, propionate, octanoate, especially from C₁₀-C₁₈ carboxylic acids, for example decanoate, dodecanoate, tetradecanoate, hexadecanoate, octadecanoate and oleate, alkylpolyethercarboxylate, alkylsulphonate, arylsulphonate, alkylarylsulphonate, alkylsulphate, alkylpolyethersulphate, phosphates derived from phosphoric acid mono alkyl/aryl ester and phosphoric acid dialkyl/aryl ester. The properties of the polyorganosiloxane compounds can be, inter alia, modified based upon the selection of acids used.

The above described polyorganosiloxane compounds comprising quaternary ammonium functions and ester functions may also contain: 1) individual molecules which contain quaternary ammonium functions and no ester functions; 2) molecules which contain quaternary ammonium functions and ester functions; and 3) molecules which contain ester functions and no quaternary ammonium functions. While not limited to structure, the above described polyorganosiloxane compounds comprising quaternary ammonium functions and ester functions are to be understood as mixtures of molecules comprising a certain averaged amount and ratio of both moieties.

Various monofunctional organic acids may be utilized to yield the esters. Exemplary embodiments include C₁-C₃₀ carboxylic acids, for example C₂, C₃, C₈ acids, C₁₀-C₁₈ carboxylic acids, for example C₁₂, C₁₄, C₁₆ acids, saturated, unsaturated and hydroxyl functionalized C₁₈ acids, alkylpolyethercarboxylic acids, alkylsulphonic acids, arylsulphonic acids, alkylarylsulphonic acids, alkylsulphuric acids, alkylpolyethersulphuric acids, phosphoric acid mono alkyl/aryl esters and phosphoric acid dialkyl/aryl esters.

### Additional Components

The non-aqueous base composition of the present invention may include other additional components, which may be selected by the artisan according to the desired characteristics of the final product and which are suitable for rendering the composition more cosmetically or aesthetically acceptable or to provide them with additional usage benefits. Such other additional components generally are used individually at levels of 0.001% to 10%.

A wide variety of other additional components can be formulated into the present compositions. These include: other conditioning agents such as hydrolysed collagen with tradename Peptein 2000 available from Hormel, vitamin E with tradename Emix-d available from Eisai, panthenol available from Roche, panthenyl ethyl ether available from Roche, hydrolysed keratin, proteins, plant extracts, and nutrients; preservatives such as benzyl alcohol, methyl paraben, propyl paraben and imidazolidinyl urea; pH adjusting agents, such as citric acid, sodium citrate, succinic acid, phosphoric acid, sodium hydroxide, sodium carbonate; coloring agents, such as any of the FD&C or D&C dyes; perfumes; ultraviolet and infrared screening and absorbing agents such as benzophenones; and antidandruff agents such as zinc pyrithionc.

### Method of Preparation of Product Compositions

The product composition of the present invention can be prepared by, for example, below METHOD A or METHOD B.

### Method A

Prepare a mixture composition comprising a surfactant and a high melting point fatty compound and a benefit agent, for example, as described in more detail below. Separately prepare a non-aqueous base composition comprising at least 75% of a fluid that is not a hydrogen donor, for example, as described below. Mix the mixture composition and the non-aqueous base composition, to form a discrete particle of the mixture composition dispersed in the non-aqueous base composition. When mixed, the mixture composition and the non-aqueous base composition respectively have a temperature lower than the melting point of the high melting point fatty compound. When mixed, the mixture composition has a temperature lower than the melting point of the high melting point fatty compound contained in the mixture composition, and the mixture composition has such temperature during and after mixing with the non-aqueous base composition. The non-aqueous base composition also has a temperature lower than the melting point of the high melting point fatty compound contained in the mixture composition when mixed with the mixture composition and has such temperature during and after mixing with the mixture composition. Thus, when mixed, the mixture composition and the non-aqueous base composition respectively have a temperature of at least 2° C. lower (e.g., 5° C. lower, 10° C. lower, or 15° C. lower) than the melting point of the high melting point fatty compound. When mixed, the mixture composition and the non-aqueous base composition respectively have a temperature of from about 0° C. to about 50° C. (e.g., 10° C. to 40° C. or 15° C. to 35° C.).

### Method B

Prepare a mixture composition comprising a surfactant and a high melting point fatty compound and a benefit agent, for example, as described in more detail below. Separately prepare a non-aqueous base composition comprising at least 75% of a fluid that is not a hydrogen donor, for example, as described below. Mix the discrete particle and the non-aqueous base composition, to disperse the discrete particle in the non-aqueous base composition. The discrete particle may be solid. A solvent or carrier may be used to prepare discrete particle of the mixture composition. Such solvents and carriers are considered as components of the non-aqueous base composition when determining amounts of ingredients. When mixed, the discrete particle and the non-aqueous base composition respectively have a temperature lower than the melting point of the high melting point fatty compound. When mixed, the discrete particle has a temperature lower than the melting point of the high melting point fatty compound contained in the discrete particle, and the discrete particle has such temperature during and after mixing with the non-aqueous base composition. Also preferably, the non-aqueous base composition also has a temperature lower than the melting point of the high melting point fatty compound contained in the discrete particle when mixed with the discrete particle, and has such temperature during and after mixing with the discrete particle. When mixed, the discrete particle and the non-aqueous base composition respectively have a temperature of preferably at least 2° C. lower (e.g., 5° C. lower, 10° C. lower, or 15° C. lower) than the melting point of the high melting point fatty compound. When mixed, the discrete particle and the non-aqueous base composition respectively have a temperature of 0° C. to 50° C. (e.g., 10° C. to 40° C. or 15° C. to 35° C.)

### Preparation of the Mixture Composition

Both in METHOD A and B, the mixture composition is prepared by preparing a mixture composition comprising the surfactant and the high melting point fatty compound, wherein the temperature of the mixture composition is higher than the melting point of the high melting point fatty compound contained in the mixture composition, and then cooling the mixture composition to a temperature which is lower than the melting point of the high melting point fatty compound. In METHOD B, the discrete particle can be prepared concurrently when preparing the mixture composition during the above cooling step, or can be prepared after forming the mixture composition.

The temperature of the melted mixture composition is at least 2° C. (e.g., 5° C., 10° C., or 15° C.) higher than the above melting point of the high melting point fatty compound. The temperature of the melting mixture composition can be 30° C. to 150° C. (e.g., 40° C. to 100° C., 50° C. to 95° C., 55° C. to 90° C., or 66° C. to 90° C.). The molten mixture composition is cooled to a temperature which is lower than a melting point of the high melting point fatty compound contained in the mixture composition, i.e., at least 2° C. lower than the melting point of the high melting point fatty compound contained in the mixture composition. In some instances, the mixture composition may be cooled to a temperature of-200° C. to about 50° C. (e.g., −40° C. to 50° C. or 0° C. to about 30° C.).

### When Containing the Benefit Agents in the Mixture Composition

When the mixture composition further comprises the benefit agents, the mixture composition can be prepared by steps of:

Preparing a melting mixture composition comprising the surfactant and the high melting point fatty compound, wherein the temperature of the melting mixture composition is higher than a melting point of the high melting point fatty compound contained in the mixture composition;

Cooling the melting mixture composition to the temperature which is lower than a melting point of the high melting point fatty compound contained in the mixture composition, to form the mixture composition, wherein the benefit agent can be added anytime depending on the properties of the benefit agent, for example, the benefit agent can be added to the mixture composition before cooling, during cooling especially when using volatile benefit agent such as perfumes, or after cooling preferably right after cooling such as within 30 min after cooling.

When the mixture composition comprises a benefit agent, the benefit agent can be homogeneously mixed with the mixture composition, and homogeneous discrete particles can be formed in the compositions.

Alternatively, in the discrete particle, the benefit agent can form an inner core covered by an outer shell formed by the mixture composition.

### When Containing the Benefit Agent in the Non-Aqueous Base Composition

When the non-aqueous base composition comprises a benefit agent, the benefit agent can be homogeneously mixed with the non-aqueous base composition.

When the non-aqueous base composition comprises a benefit agent, the benefit agent can be added to the non-aqueous base composition anytime, for example, before adding the mixture composition and/or the discrete particle, after adding the mixture composition and/or the discrete particle, and/or concurrently with the discrete particle.

### Test Methods

### 4 Week Stability Test:

This is a Test Method for Stability, that is, for determining failure/success due to disintegration.

0.2 g of the discrete particles are added to 99.8 g of the non-aqueous base and placed in a temperature-controlled room, at 40 degrees Celsius. The appearance of the non-aqueous base and discrete particles is checked after 4 weeks. The mixture should be placed in a see-through container to aid the assessment.

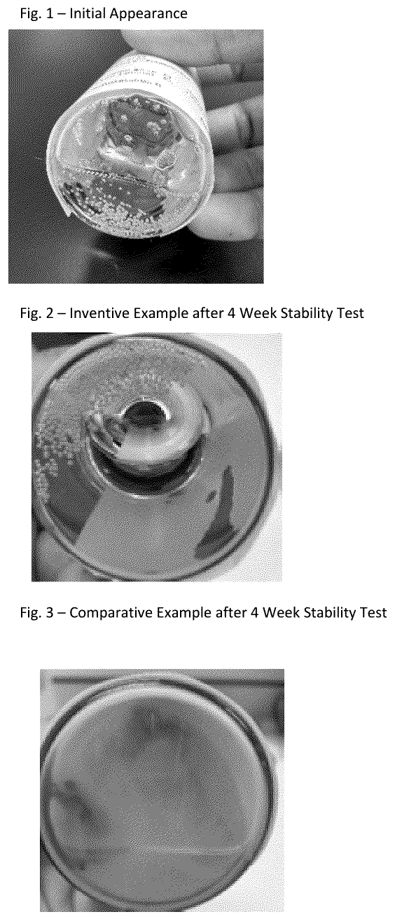

FIG. 1 is an example of the initial appearance of a sample that has been successfully formulated, i.e., forms discrete particles within the non-aqueous base. FIG. 1 is an initial photo (i.e., right after making the example) of inventive example 3, however it is a good representation of any of the initial example appearances, both inventive and comparative.

FIG. 2 is an example of an inventive composition, as it has stable discrete particles after 4 weeks under the 4 Week Stability Test. After four weeks, the discrete particles remain discrete particles with no disintegration. To be considered stable, all particles should remain discrete and intact. The particles may change in their absolute size or volume but should still remain discrete. FIG. 2 is a photo of Inventive Example 5 after 4 weeks under the 4 Week Stability Test.

FIG. 3 shows an example of disintegration of discrete particles after 4 weeks under the 4 Week Stability Test. This comparative example, while initially formulated with discrete particles, did not remain stable and the discrete particles disintegrated and/or dissolved. FIG. 3 is a photo of Comparative Example 1, taken after 4 weeks under the 4 Week Stability Test.

For non-inventive or comparative examples, the presence of a hydrogen donor within the non-aqueous base composition leads to higher polarity and thus higher interaction and compatibility between the discrete particle and the non-aqueous base composition. The higher compatibility and interaction eventually lead to disintegration or dissolving of the non-aqueous composition in a “like dissolves like” concept. Thus, stability is achieved when the non-aqueous base comprises at least about 75% of a non-hydrogen donor fluid.

### ALogP:

ALogP is the Octanol/Water partition coefficient that describes the hydrophilicity or hydrophobicity of the molecule. “AlogP” as used herein, is an identification of the octanol-water partition coefficient of an active. Ghose and Crippen used this atom-based method to calculate the octanol-water partition coefficient (log P), and the molar refractivity (MR) for incoming molecules. Log P provides a measure of the hydrophobicity of the molecule, while MR contains information about molecular volume and polarizability. AlogP is calculated herein using Pipeline Pilot software (Biovia™) ver 9.2.

### Method of Preparation of the Product Composition

The embodiments disclosed and represented in Table 3 are hair conditioning product compositions of the present invention, and were prepared by Method B explained above, and the following in more detail:

Preparing the mixture composition comprising the surfactant and the high melting point fatty compound and benefit agent, wherein the mixture composition was prepared by the following method:
Preparing a melting mixture composition wherein the temperature of the melting mixture composition is higher than a melting point of the high melting point fatty compound contained in the mixture composition, i.e., from about 66° C. to about 90° C.;
Cooling the melting mixture composition to a temperature which is lower than a melting point of the high melting point fatty compound contained in the mixture composition, i.e., from about 0° C. to about 40° C., to form the mixture composition;
Preparing a solid discrete particle consisting of the mixture composition;
Separately preparing a non-aqueous base composition;
Mixing the discrete particle and the non-aqueous base composition, both having a temperature lower than a melting point of the high melting point fatty compound contained in the mixture composition, i.e., from about 10° C. to about 40° C., so that the discrete particle is dispersed in the non-aqueous base composition.

The hair conditioner product compositions disclosed in Table 4 are comparative examples and were prepared by the above METHOD B.

### Examples

Table 1 below shows various materials and their ALogP and number of hydrogen donors. As can be seen in Tables 1-4, when a material has satisfied both conditions of not being a hydrogen donor and having an AlogP of at least 6, the non-aqueous product composition is able to maintain the solid discrete particles for at least 4 weeks at 40° C. For clarity, a fluid that does not comprise any hydrogen donor groups is a fluid that is not a hydrogen donor.

TABLE 1
Materials and their ALogP and Number of Hydrogen Donors

| | | | Has both 0 H donation | Solid Discrete particles |
|---|---|---|---|---|
| | AlogP | H Donor | and AlogP of at least 6 | visible after 4 weeks 40° C. |
| 1-Decanol | 3.71 | 1 | NO | NO |
| Hexyldecanol | 6.43 | 1 | NO | NO |
| Oleic acid | 6.86 | 1 | NO | NO |
| Oleyl Alcohol | 6.91 | 1 | NO | NO |
| 1-Octanol | 2.80 | 1 | NO | NO |
| Isohexadecane | 7.47 | 0 | YES | YES |
| Isododecane | 6.95 | 0 | YES | YES |
| Triethylhexanoin | 8.59 | 0 | YES | YES |
| Isodecyl Oleate | 11.15 | 0 | YES | YES |
| Decyl oleate | 10.95 | 0 | YES | YES |
| Silicone Quaternium-26 | n/a | 0 | YES | YES |
| Cyclopentasiloxane | n/a | 0 | YES | YES |
| Dimethicone | n/a | 0 | YES | YES |

Tables 2-4 show the compositions (non-aqueous base and mixture composition (discrete particles)) that populate Table 1.

TABLE 2
Composition of solid discrete particles

| | A1 | B1 | C1 | D1 |
|---|---|---|---|---|
| Cetyl Alcohol | 45 | 45 | 45 | 30 |
| Stearyl Alcohol | 45 | 45 | 45 | 30 |
| BTMS | 5 | | | 35 |
| SAPDMA | | 5 | | |
| BTMAC | | | 10 | |
| Benefit Agent | 5 | 5 | | 5 |

TABLE 3
Inventive Examples of Conditioner Product Compositions

| | Inv 1 | Inv 1 | Inv 3 | Inv 4 | Inv 5 | Inv 6 | Inv 7 | Inv 8 | Inv 9 |
|---|---|---|---|---|---|---|---|---|---|
| A1 | 0.2 | 0.2 | 0.2 | 0.2 | 0.2 | | | 0.2 | 0.2 |
| B1 | | | | | | 0.2 | | | |
| C1 | | | | | | | 0.2 | | |
| 1-Decanol | | | | | | | | 24.8 | |
| Isohexadecane | | 99.8 | | | | | | | |
| Isododecane | | | | | | | | | 60 |
| Triethylhexanoin | 99.8 | | | | | 99.8 | 99.8 | 75 | |
| Isodecyl Oleate | | | 99.8 | | | | | | |
| Decyl oleate | | | | 99.8 | | | | | |
| Silicone | | | | | 99.8 | | | | |
| Quaternium-26 | | | | | | | | | |
| Dimethicone | | | | | | | | | 30 |
| Other materials | | | | | | | | | 9.8 |
| No Hydrogen | Yes | Yes | Yes | Yes | Yes | Yes | Yes | Yes | Yes |
| Donor (>75% of | | | | | | | | | |
| composition)? | | | | | | | | | |
| Does not dissolve | Yes | Yes | Yes | Yes | Yes | Yes | Yes | Yes | Yes |
| at 4 weeks 40° C. | | | | | | | | | |

TABLE 4
Conditioner product compositions for Table 1: Comparative Examples

| | Comparative | Comparative | Comparative | Comparative | Comparative | Comparative | Comparative | Comparative | Comparative |
|---|---|---|---|---|---|---|---|---|---|
| | Example 1 | Example 2 | Example 3 | Example 4 | Example 5 | Example 6 | Example 7 | Example 8 | Example 9 |
| A1 | 0.2 | 0.2 | 0.2 | 0.2 | 0.2 | | | 0.2 | 0.2 |
| B1 | | | | | | 0.2 | | | |
| C1 | | | | | | | 0.2 | | |
| 1-Decanol | 99.8 | | | | | 99.8 | 99.8 | 50 | 75 |
| Hexyldecanol | | 99.8 | | | | | | | |
| Oleic acid | | | 99.8 | | | | | | |
| Oleyl Alcohol | | | | 99.8 | | | | | |
| Triethylhexanoin | | | | | | | | 49.8 | 24.8 |
| 1-Octanol | | | | | 99.8 | | | | |
| No Hydrogen | No | No | No | No | No | No | No | No | No |
| Donor (>75% of | | | | | | | | | |
| composition)? | | | | | | | | | |
| Does not dissolve | No | No | No | No | No | No | No | No | No |
| at 4 weeks 40 C. | | | | | | | | | |

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A personal care composition comprising:
discrete particles of a mixture composition, wherein the mixture composition comprises a surfactant and a high melting point fatty compound; and
a non-aqueous base, wherein the non-aqueous base comprises at least 75 of a fluid that is not a hydrogen donor.

2. The personal care composition of claim 1, wherein the fluid has an AlogP of at least 6.

3. The personal care composition of claim 1, wherein the non-aqueous base comprises at least 90%, by weight of the base, of the fluid.

4. The personal care composition of claim 1, wherein the fluid comprises at least one of a silicone, a branched hydrocarbon, an ester, and an alkane.

5. The personal care composition of claim 4, wherein the fluid is a silicone selected from a polyalkylsiloxane, a cyclopentasiloxane, a dimethicone, an aminosilicone, and combinations thereof.

6. The personal care composition of claim 1, wherein the fluid comprises at least one of isohexadecane, isododecane, triethylhexanoin, isodecyl oleate, decyl oleate, silicone quaternium-26, dimethicone, and cyclopentasiloxane.

7. The personal care composition of claim 1, wherein the fluid has a melt point below 25° C.

8. The personal care composition of claim 1, wherein the composition is free of or substantially free of anionic surfactants, zwitterionic surfactant, and amphoteric surfactants.

9. The personal care composition of claim 1, wherein the discrete particles are not coated or encapsulated.

10. The personal care composition of claim 1, wherein the discrete particles are stable according to the 4 Week Stability Test.

11. The personal care composition of claim 1, wherein the discrete particles are solid.

12. The personal care composition of claim 1, wherein the discrete particles comprise less than about 30% of water.

13. The personal care composition of claim 12, wherein the discrete particle is anhydrous.

14. The personal care composition of claim 1, wherein the surfactant is a cationic surfactant, a nonionic surfactant, or a mixture thereof.

15. The personal care composition of claim 14, wherein the surfactant is a cationic surfactant.

16. The personal care composition of claim 1, wherein the personal care composition is a hair oil composition.

17. The personal care composition of claim 1, wherein the discrete particles further comprise a benefit agent.

18. The personal care composition of claim 17, wherein the benefit agent comprises a silicone compound, a perfume, an incompatible agent, or a combination thereof.

19. The personal care composition of claim 1, wherein the non-aqueous base comprises 100%, by weight of the base, of the fluid.
